(19) United States
(12) Patent Application Publication
(10) Pub. No.: US 2010/0198305 A1
(43) Pub. Date: Aug. 5, 2010

(54) DETECTION OF IMPLANT FUNCTIONALITY

(75) Inventor: John Michael Farbarik, Castro Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pavad Medical, Inc., Fremont, CA (US)

(21) Appl. No.: 12/696,463

(22) Filed: Jan. 29, 2010

Related U.S. Application Data
(60) Provisional application No. 61/148,890, filed on Jan. 30, 2009.

(51) Int. Cl. A61N 1/08

(52) U.S. Cl. 607/60

(57) ABSTRACT

Techniques are disclosed for detecting a functionality of a medical device implanted within the human body. A testing method includes delivering power to and measuring an electrical response of ion exchange polymer metal composite (IPMC) material that is part of the implant device in a first testing interval. Power can be transmitted at radio frequencies which penetrate tissue such that direct contact with the implant device is not required. Following the first testing interval, the implant can be powered continuously for a predetermined time. The implant can be powered in a second testing interval and the electrical response can again be measured. A functionality of the implant device can be detected based on the electrical response in the first and second testing intervals. The testing method can be practiced in connection with a handheld device, a retainer, or other suitable apparatus.

This application claims the benefit of and is a non-provisional of U.S. Provisional Application 61/148,890 filed on Jan. 30, 2009 (Atty. Docket No. 026705-002100US), which is assigned to the assignee hereof and incorporated herein by reference for all purposes.

### BACKGROUND

The present invention relates generally to ion exchange polymer metal composite (IPMC) materials and, more specifically, to detecting a functionality of IPMC-based devices implanted within the human body.

Snoring is very common among mammals including humans. Snoring is a noise produced while breathing during sleep due to the vibration of the soft palate and uvula. Not all snoring is bad, except it bothers the bed partner or others near the person who is snoring. If the snoring gets worse over time and goes untreated, it could lead to apnea.

Those with apnea stop breathing in their sleep, often hundreds of times during the night. Usually apnea occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes it to be narrowed.

The specific therapy for sleep apnea is tailored to the individual patient based on medical history, physical examination, and the results of polysomnography. In some cases, a medical device is implanted within the patient's body for controlling the airway passage. Such devices can reduce the frequency and/or severity of apneic events. Generally speaking, in-vivo medical devices are not readily accessible for testing and/or observation.

### BRIEF SUMMARY

Techniques are disclosed for detecting a functionality of a medical device implanted within the human body. A testing method includes delivering power to and measuring an electrical response of ion exchange polymer metal composite (IPMC) material that is part of the implant device in a first testing interval. Power can be transmitted at radio frequencies which penetrate tissue such that direct contact with the implant device is not required. Following the first testing interval, the implant can be powered continuously for a predetermined time. The implant can be powered in a second testing interval and the electrical response can again be measured. A functionality of the implant device can be detected based on the electrical response in the first and second testing intervals. The testing method can be practiced in connection with a handheld device, a retainer, or other suitable apparatus.

In some embodiments, power is withheld from the IPMC device for a predetermined time before testing is initiated. Power can be delivered with a series of pulses during the first and second testing intervals. Detecting the functionality of the IPMC device can include comparing the electrical response measured in the first testing interval with the electrical response measured in the second testing interval. Detecting the functionality of the IPMC device can also include identifying specific abnormalities of the IPMC device. In one embodiment, the method includes indicating the functionality of the IPMC device when the electrical response measured in the first testing interval is larger than the electrical response measured in the second testing interval. The method can also include displaying information about the functionality of the IPMC device at a user interface such as with one or more light emitting diodes (LEDs) and/or a display panel.

In one embodiment, a device for testing an ionic polymer-metal composite implant is disclosed. The device includes a transducer configured to supply power to the implant when the implant is disposed within a patient's body and a detector configured to measure an electrical response of the implant. A controller is coupled to the transducer and the detector and controls their respective operations. The controller is configured to perform a test of the implant by supplying power to and measuring the electrical response of the implant in testing intervals. The controller can be configured to detect a functionality of the implant by performing a first test, powering the implant continuously, and performing a second test after the implant is continuously powered. Power can be supplied with a series of pulses in each of the testing intervals and the electrical response can include a voltage field in the vicinity of the implant. In some embodiments, the controller detects the functionality based on a comparison of the electrical response in the first test and the electrical response in the second test. The device can detect abnormalities of the implant and can also include user interface elements for displaying information about the implant functionality.

For a further understanding of the nature and advantages of the invention, reference is made to the following description taken in conjunction with the accompanying figures. It is to be expressly understood, however, that each of the figures is provided for the purpose of illustration and description only and is not intended as a definition of the limits of the embodiments of the present invention.

### BRIEF DESCRIPTION OF THE DRAWINGS

FIG. 1 shows one embodiment an implant device with IPMC material for testing.

FIG. 2 is a front view of the implant device depicted in FIG. 1.

FIG. 3 shows a further embodiment of an IPMC implant device.

FIG. 4 shows aspects of implant functionality.

FIG. 5 shows an implant testing apparatus.

FIG. 6 is a flowchart of an implant testing method.

FIGS. 7A-7B show an electrical model and response characteristic of IPMC material.

FIG. 8 shows a block diagram of an implant testing device.

FIG. 9 shows a handheld testing device for detecting a functionality of an implant.

FIG. 10A shows a retainer-based testing device for detecting a functionality of an implant within the human body.

FIG. 10B shows the retainer of FIG. 10A in relation to an airway implant device.

FIG. 11 shows a base unit for processing data collected by the retainer of FIG. 10.

The features, objects, and advantages of embodiments of the disclosure will become more apparent from the detailed description set forth below when taken in conjunction with the drawings, in which like elements bear like reference numerals.

### DETAILED DESCRIPTION OF EMBODIMENTS

Applicants' co-pending U.S. patent applications Ser. No. 12/192,924 (Attorney Docket No. 026705-001100US) and Ser. No. 12/205,625 (Attorney Docket No. 026705-001200US) provide further details of medical devices with which the present invention can advantageously be utilized and each is incorporated herein by reference for all purposes. Although described herein with reference to airway implant devices, the present disclosure is generally applicable to any device and/or diagnostic method involving the detection of IPMC functionality and power delivery.

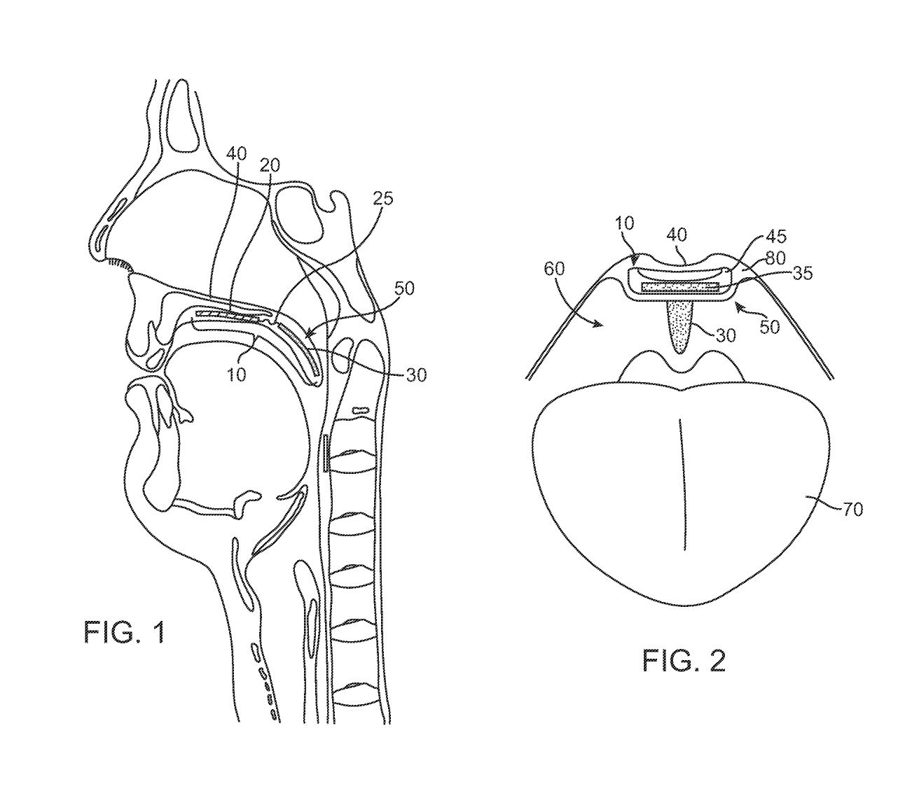

FIG. 1 illustrates an airway implant device 10 for which a functionality is detected according to embodiments of the present invention. A cross-sectional view of a patient is shown in which the implant device 10 is inserted into the body for modulating the opening of the airway passage. As shown, implant device 10 includes a controller 20 and a deformable element 30. The controller 20 can be inserted into the hard palate 40 whereas the deformable element 30 can be implanted in the uvula or soft palate 50. Generally, the in-vivo implant device is disposed beneath tissue and is not directly accessible for testing.

Controller 20 can supply power to the deformable element 30 via a conducting member 25. Alternatively, the controller can be integrated with the deformable member. The controller 20 may also include an inductor for receiving power from an external source. For example, a patient can wear a non-implanted device such as a mouthpiece, retainer, or patch which includes power transmit circuitry for sourcing RF power to the in-vivo implant 10. An inductor included with the implant 10 can be configured to convert the RF energy into electricity which can be delivered to the deformable element 30 via the conducting member 25. Other power transfer mechanisms can also be used.

Deformable element 30 includes an IPMC material. The IPMC material can have a base polymer embedded, or otherwise appropriately mixed, with a metal. The IPMC base polymer can be perfluoronated polymer, polytetrafluoroethylene, polyfluorosulfonic acid, perfluorosulfonate, polyvinylidene fluoride, hydrophilic polyvinylidene fluoride, polyethylene, polypropylene, polystyrene, polyaniline, polyacrylonitrile, cellophane, cellulose, regenerated cellulose, cellulose acetate, polysulfone, polyurethane, polyvinyl alcohol, polyvinyl acetate and polyvinyl pyrrolidone, or combinations thereof. The IPMC metal can be platinum, gold, silver, palladium, copper, carbon, or combinations thereof.

The IPMC material of deformable element 30 is energized by charge or current from the controller 20. The energized IPMC material deforms in shape and thereby alters the airway passage so that, when energized, the deformable element 30 modulates the airway opening in its immediate vicinity. In a non-energized state, the IPMC material relaxes and conforms to the surrounding airway. As a result, when power is removed, deformable element 30 is flexible and/or soft. As described herein, embodiments of a testing system, apparatus, and method detect a functionality of the IPMC material which forms part of the in-vivo implant.

FIG. 2 is a front view illustrating a relation of implant device 10 to the airway passage as well as to the mouth 60 and tongue 70. As shown, implant device 10 can be inserted such that a housing 45 of controller 20 is within the periosteum 80 inferior to the ridge of the hard palate 40, with the deformable element 30 extending into the soft palate 50. An inductor 35 within housing 45 can be configured to receive an inductive power transfer. As shown in FIG. 1 and FIG. 2, some or all of the implant device is disposed beneath the tissue such that there is a limited ability to observe and test its operation using non-invasive procedures.

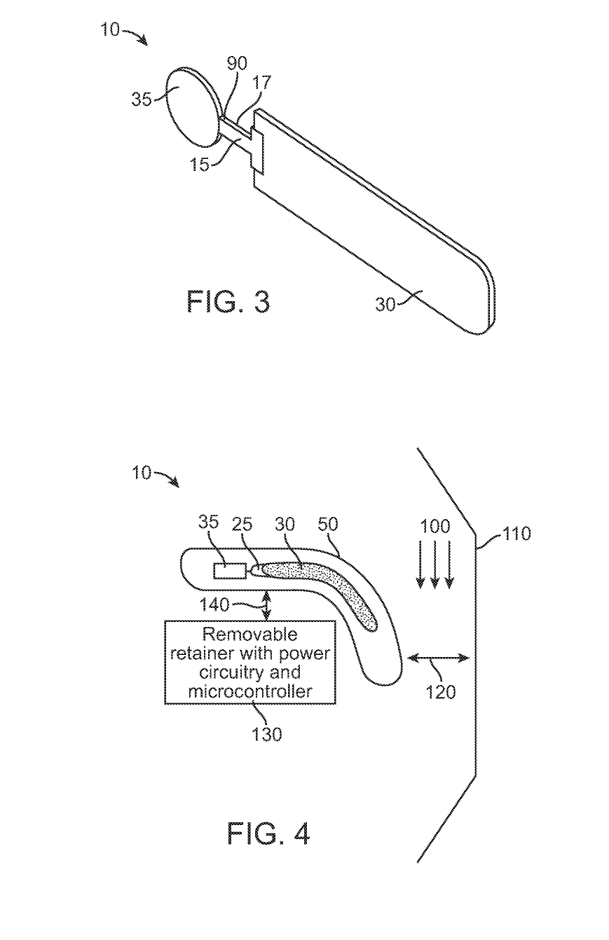

FIG. 3 illustrates an airway implant device 10 for testing in accordance with embodiments of the present invention. The device of FIG. 3 includes an IPMC actuator element 30 connected to an anode 15 and cathode 17 and also to an induction coil 35. The device also includes a controller 90, such as a microprocessor. Circuitry within the controller is not shown. The controller 90 can be configured to pick up AC signals from the induction coil 35 and convert them to DC current. The controller 90 can also include a time delay circuit and/or a sensor. The sensor can be configured to sense the collapsing and/or narrowing of the airway passage and cause the implant to energize the IPMC actuator 30. In this way, the implant device 10 can completely or partially open up the airway in which it is implanted. In some embodiments, the controller 90 can also gather and transmit data relating to IPMC-actuator 35 such as a change in its electrical properties.

FIG. 4 shows a further embodiment of an airway implant device 10 for functionality testing. As illustrated, air flow 100 passes along laryngeal wall 110 to the lungs. In an unobstructed breathing cycle, a distance 120 between the soft palate 50 and laryngeal wall 110 permits a normal air flow in the airway passage. However, during an apneic event, movement of the soft palate 50 can block the airway passage and disrupt breathing.

In the present embodiment, the patient wears a non-implanted device 130 for operating the airway implant. The non-implanted device 130 can be a retainer or mouthpiece that includes power circuitry for sourcing RF energy to the airway implant device 10. As shown, the inductor 35 converts the RF energy into electricity and delivers an electric current to the IPMC material of deformable element 30 via conductor 25. In response to the current flow, the IPMC material within soft palate 50 deforms and increases the distance 120 between soft palate 50 and laryngeal wall 110. The non-implanted device 130 can collect data associated with operating the implant 10 and/or it can receive data gathered by the airway implant 10.

The ability to effectively modulate the airway passage with the implant device can depend upon proper functioning of the IPMC material. A number of factors can affect IPMC functionality. For example, the IPMC material can be damaged during the implant procedure such as by tearing the deformable element or severing it from the controller. Similarly, the controller and/or conducting members can be damaged such that they do not supply a proper activation current to the IPMC material. The IPMC material can also change over time with consequent changes in its electrical properties. For these and other reasons, embodiments of the present invention are directed to capabilities of detecting the functionality of the IPMC material and verifying the power transfer capability of the implant device.

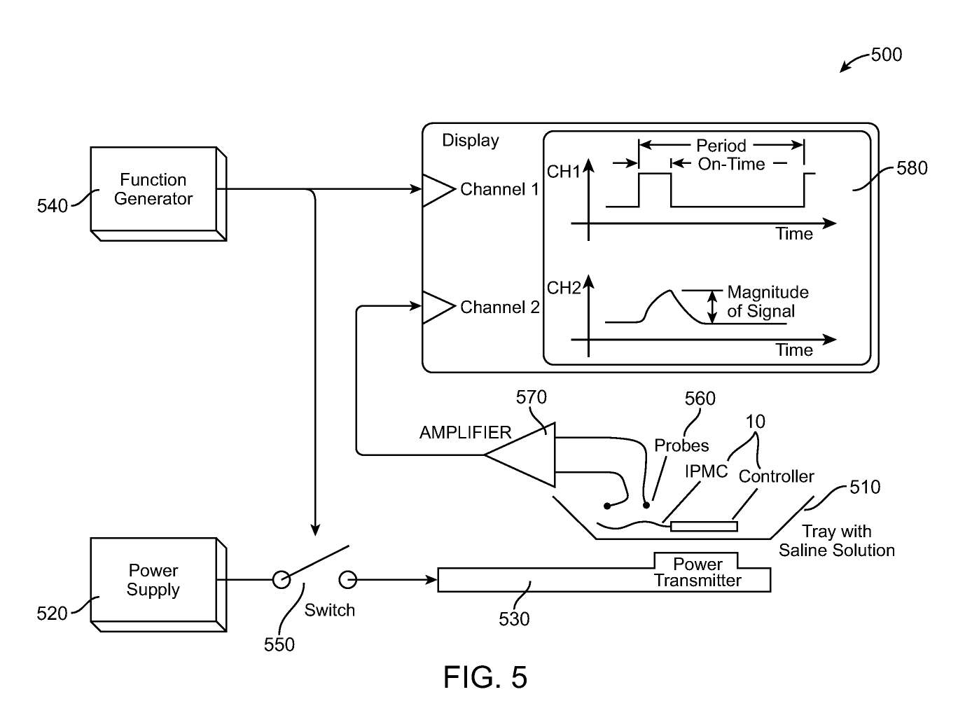

FIG. 5 shows an embodiment of an implant testing apparatus. For purposes of discussion, an in-vitro test apparatus 500 is illustrated. However, it will be understood that a testing method and apparatus according to embodiments of the present invention are intended to be used to detect a functionality of in-vivo devices. In other words, the testing apparatus and method steps detect a functionality of an implant that is inserted into a patient's body.

As shown, test apparatus 500 includes an implant device 10 which is to be tested, a power supply 520, and a transmitter 530. Power supply 520 is coupled to the transmitter 530 by a switch 550. In this embodiment, power supply 520 is configured to source an alternating current to transmitter 530 under the control of switch 550. Transmitter 530 can include an induction coil for converting the alternating current to RF energy corresponding to power delivery to an in-vivo implant.

Implant device 10 is situated in a tray 510 and positioned at a distance from transmitter 530 which may correspond to the separation between a non-implanted control device and an in-vivo implant. For example, the distance between transmitter 530 and implant device 10 can be similar to the separation distance 140 between mouthpiece 130 and implant 10 as illustrated in FIG. 4. Tray 510 can include a saline solution that covers the implant 110 and corresponds to a patient's body chemistry near the site of the implant.

A function generator 540 is coupled to the switch 550 and controls its operation. The function generator 540 can be configured to generate different waveforms which are appropriate to the implant under test. When function generator 540 closes switch 550, power is delivered from power supply 520 to transmitter 530. Transmitter 530 sources RF energy to implant device 10 which can include a second transducer and related circuitry for converting the RF energy into a DC voltage. The DC voltage can be delivered to the IPMC material for activating the implant.

Test apparatus 500 also includes detection circuitry 560, 570 and a display unit 580. The detection circuitry can include probes 560 and an amplifier 570. For testing, probes 560 can be situated near to the implant device 10 and configured to measure a DC voltage field. As an example, probes 560 can be wires which are suspended in the saline solution of tray 510 near to the IPMC material. Note that, as with in-vivo testing, it is not necessary for probes 560 to make contact with the IPMC material itself Instead, probes 560 can measure a surrounding voltage field and can therefore be used to test the functionality of an implant that has been inserted into the human body and which is not directly accessible.

As shown, probes 560 are coupled to amplifier 570. Amplifier 570 can be an instrumentation amplifier or a different amplifier which scales the probe measurements to a level suitable for viewing on display 580. By way of illustration, amplifier 570 can be a model AD8221 from Analog Devices, Inc. Display 580 can be an oscilloscope or liquid crystal panel configured to provide a visual representation of the DC voltage field measurements.

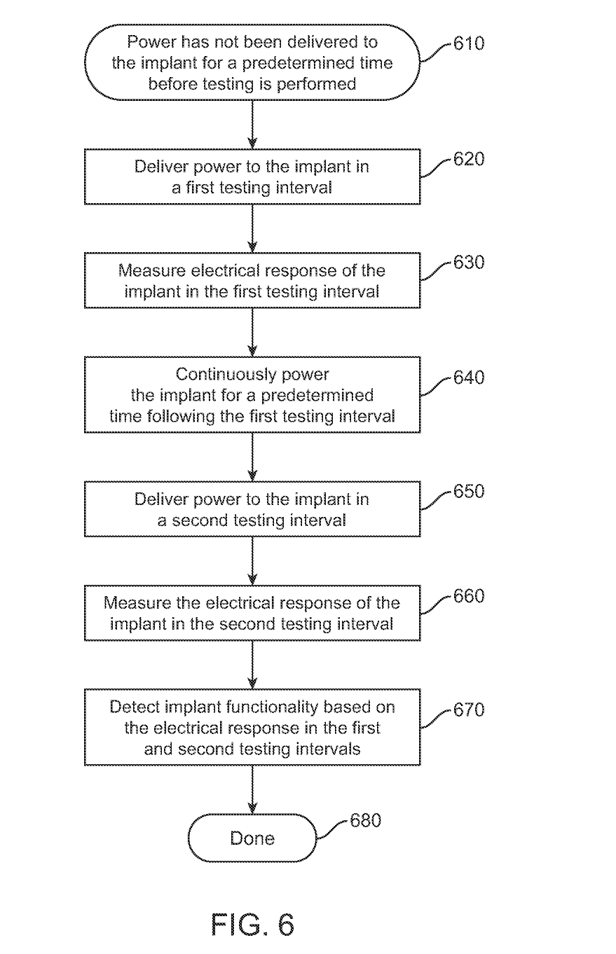

The operation of test apparatus 500 is now described in connection with FIG. 6. At the start of the testing process 610, the implant is in a de-energized state. To reduce the potential for carryover effects, the implant can be maintained in a de-energized state for a predetermined time before testing is initiated. For example, the implant can be de-energized for at least one hour before testing is performed.

At block 620, power is delivered to the implant in a first testing interval. For example, function generator 540 can be configured to produce a series of short-duration pulses for opening and closing switch 550. Operating switch 550 causes power to be delivered to the implant device 10 from power supply 520. The pulse waveform and duration can be chosen to facilitate signal processing operations associated with measuring the implant's response to the power transfer. Power supply 520 can be configured to deliver power at a level that is consistent with in-vivo operation of the implant device 10. Alternatively, power supply 520 can source power at different levels, or can change levels during the first testing interval.

In an exemplary embodiment, function generator 540 is configured to produce a series of 50 ms pulses every 1000 ms as illustrated in the CH1 portion of display 580. A total of 10-20 pulses can be generated during the first testing interval to accomplish a burst-powering of the implant device. A variety of waveforms can be used for delivering power to the implant in the first testing interval and that the present invention is not limited to a particular waveform or sequence.

At block 630, an electrical response of the implant is measured in the first testing interval. The electrical response can include a magnitude of a DC voltage field surrounding the IPMC material. For example, function generator 540 can be configured to synchronize sampling of the output signal of amplifier 570 so that the measurements obtained by probes 560 during delivery of power to the implant device 10 can be reproduced on display 580 (CH2). With one exemplary IPMC device in normal operating condition, a DC field magnitude of approximately 100-150 mV can be measured in response to the burst-powering.

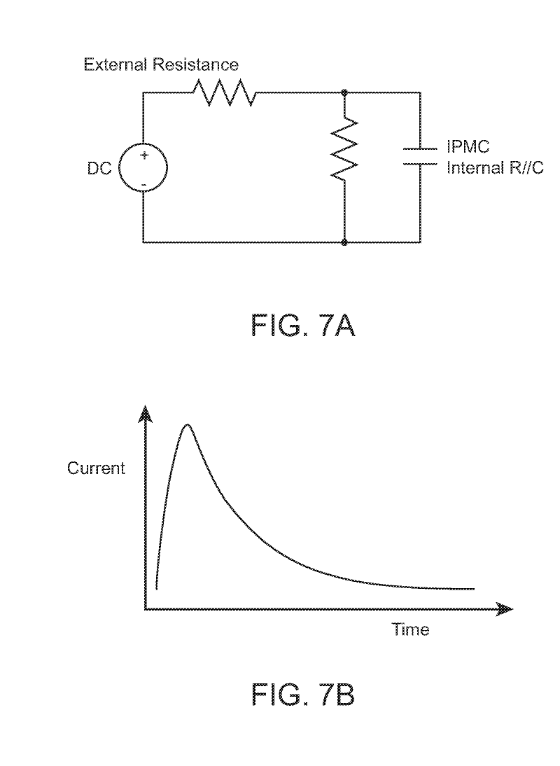

It has been discovered that the IPMC material of the implant device exhibits a capacitive response to burst-powering. FIGS. 7A-7B provide an illustration of the electrical response characteristics of an exemplary IPMC material. As shown in FIG. 7A, the implant device can be modeled as a DC voltage source coupled to a resistive-capacitive load. The voltage source can be provided by the control portion of the implant device which, for example, can include an induction coil and voltage regulation circuitry. The deformable IPMC element is modeled with an external resistance and a parallel internal resistance and capacitance. When a step voltage is applied, the IPMC material can exhibit an in-rush characteristic similar to the first-order circuit of FIG. 7A.

FIG. 7B illustrates the in-rush characteristic associated with IPMC material in normal operating condition. As shown, when a DC voltage is applied, a high initial current flows in the external resistance. As current continues to flow, the internal capacitance of the IPMC material is charged. With this charging phenomena, as the capacitive voltage increases with time, current flow in the external resistance decreases. A voltage field corresponding to the inrush current of

FIG. 7B is produced in the saline solution around the IPMC material. In accordance with the present invention, a magnitude of the voltage field is measured and used to provide an indication of implant functionality.

Returning to FIGS. 5-6, when the first testing interval is complete and the electrical response to the power transmission has been measured, block 640, the implant device is then powered continuously for a predetermined time. For example, function generator 540 can be programmed to close switch 550 for approximately one minute or longer as needed to fully energize the IPMC material of the implant device. Instead of continuous power delivery, power can be delivered to the implant through a series of longer-duration pulses or similar waveforms sufficient to fully charge the internal capacitance of the IPMC material.

At block 650, after the continuous powering, a second testing interval is initiated for burst-powering the implant device. The second testing interval can be substantially similar to the first testing interval to permit a direct comparison of response characteristics or it can be different. For example, during the second testing interval, function generator 540 can again produce a series of 10-20 pulses having an on-time of about 50 ms and a repetition period of around 1000 ms. At block 660, the amplifier 570 output signal is sampled in the second testing interval to obtain additional measurements of the electrical response.

At block 670, measured values from each testing interval are processed. Using measurements of the electrical response, test apparatus 500 detects the functionality of the IPMC material and also verifies the delivery of power within the implant device. For example, testing by the inventor of the present application has demonstrated that under normal operating conditions, exemplary IPMC material can generate a DC voltage field with a magnitude of approximately 100-150 mV when subjected to the first testing interval. The peak magnitude of the DC voltage field (corresponding to the in-rush characteristic) also provides an indication of the power transfer capability of the implant device.

Continuously powering the implant effectively “charges” the internal capacitance of the IPMC material. Under normal operating conditions, it has been observed that burst-powering the implant in its charged state results in a decreased electrical response. For example, with some exemplary devices, the magnitude of the DC voltage field measured during the second testing interval can be approximately 50 mV. By comparing the electrical response of the implant to the first and second testing intervals, a functionality of the IPMC material is detected. In particular, it is possible to compare the measured response values with the capacitive response of a normally functioning IPMC material. Advantageously, the entire testing procedure can be performed without direct contact with the implant device and is therefore suitable for in-vivo testing of implant functionality.

Implant functionality testing can also detect problems with the implant device. For example, a reduced peak current could indicate an increased external resistance associated with surface corrosion or oxidation of the implant device. Abnormal values of peak current may indicate a degradation or change of the IPMC material which can result from aging of the implant. Increased current levels can indicate a short circuit either at the IPMC material or between the conductors which link it with the controller. A lack of current, on the other hand, can be an indication that the IPMC material is not properly attached or that the controller is failing to deliver power.

In some embodiments, analysis of the measurements is performed to detect a type of problem with the implant. Curve fitting and signal analysis techniques can be used to generate a response profile. The response profile can then be compared to a catalog of characteristics associated with IPMC functionality. The results of the comparison can be presented to a user to facilitate problem diagnosis. For example, if a reduced peak current is detected, the response profile can be compared with profiles of IPMC material having surface oxidation as well as profiles of IPMC material at different stages of the aging process. In this way, the most likely source of the problem can be identified.

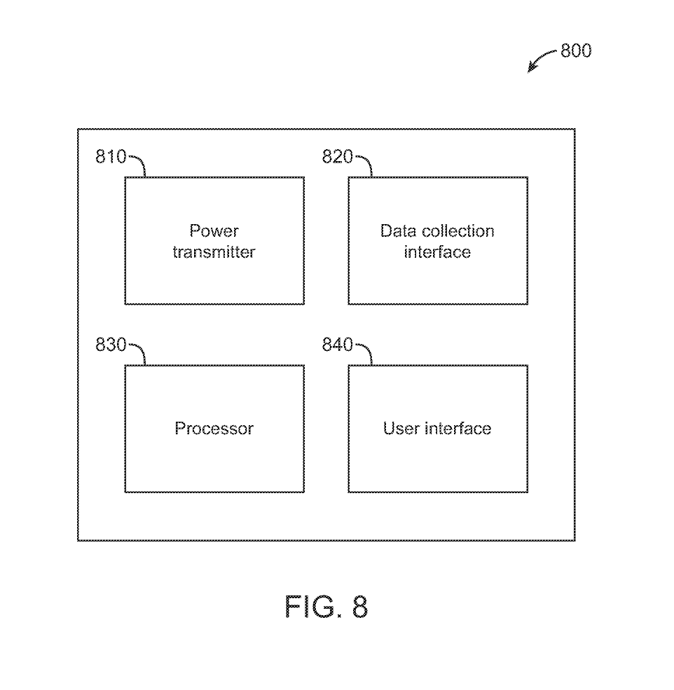

FIG. 8 is a functional block diagram of an in-vivo testing device 800 according to one embodiment of the present invention. Testing device 800 can be used to detect functionality of an IPMC material and to verify power delivery of an implant located within the human body. Functionality testing can be performed without direct contact with the implant device and without removing the implant from the patient. Testing device 800 can be embodied as a handheld device, a mouthpiece or retainer, or other non-implanted arrangement suitable for interacting with the in-vivo implant.

As shown, testing device 800 includes power transmitter 810, data collection interface 820, processor 830, and user interface 840 elements. Power transmitter 810 can include an induction coil configured to source RF energy to the implant. The RF energy can pass through the patient's body and be received by the implant device for supplying power to the IPMC material. Data interface 820 includes hardware for detecting a response of the implant to RF energy supplied by power transmitter 810. In some embodiments, data interface 820 includes sensors which contact the patient's body at a location near to the implant device. For example, the sensors can measure a DC voltage field surrounding the IPMC material in a manner that is similar to the detection circuitry 560, 570 discussed in connection with FIG. 5.

In alternative embodiments, data interface 820 includes hardware elements for receiving a communication signal with information representative of the implant's electrical response. In such embodiments, the IPMC's electrical response can be measured at the implant device and then transmitted to the testing device 800. For example, measured values of the DC voltage field can be transmitted by modulating the frequency and/or amplitude of an RF signal, or by flashing a light source or some other means. The light source can be a laser or a light emitting diode. In one embodiment, the implant device is configured to transmit measurement data by modulating emissions from a red LED, and data interface 820 includes a photodetector for receiving the modulated light signal.

Processor 830 can be a microprocessor, microcontroller, field programmable gate array (FPGA), application-specific integrated circuit (ASIC), or other programmable logic element configured to control operation of the testing device 800. In some embodiments, processor 830 is configured to implement the testing method described in FIG. 6 by controlling power transmitter 810 so as to create a first testing interval, a continuous power interval, and a second testing interval while measuring or otherwise receiving data representative of the IPMC electrical response.

As an alternative to data interface 820, processor 830 can be configured to measure a current flow in series with power transmitter 810 as a means of detecting the IPMC response characteristic. For example, a current measurement can be taken at power transmitter 810 in series with the power supply and the current flow before, during, and after the testing intervals can be recorded. Based on the level of current, the in-rush characteristic of the IPMC material can be gauged and potential problems can be identified.

Processor 830 can also be configured to perform signal processing operations on the measurements obtained from data interface 820. For example, processor 830 can synchronize the timing of the data collection with the power supplied in each testing interval and perform correlation and signal averaging to identify the IPMC electrical response in a noisy RF environment. Processor 830 can also perform statistical analysis and curve fitting operations on the measured data. In some embodiments, data gathered from the implant is stored in a memory of the testing device 800 and can be used to detect changes in implant functionality that can occur over an extended time.

In some embodiments, information about implant functionality is presented at user interface 840. For example, hand-held embodiments of the testing device 800 can include various audible and/or visual indicators for providing information about the implant. In various embodiments, light emitting diodes or a buzzer can be used to present high-level information such as whether or not normal implant function has been detected. A display panel can provide more in-depth diagnostic information such as an error code resulting from analysis of the measurement data. A user interface 840 provided with a mouthpiece or retainer embodiment can include a transmitter or data bus for sending or downloading the measurement data. For example, a retainer may be configured for serial communication with a computer and/or docking station. Many alternative user interfaces 840 are possible within the scope of the present invention.

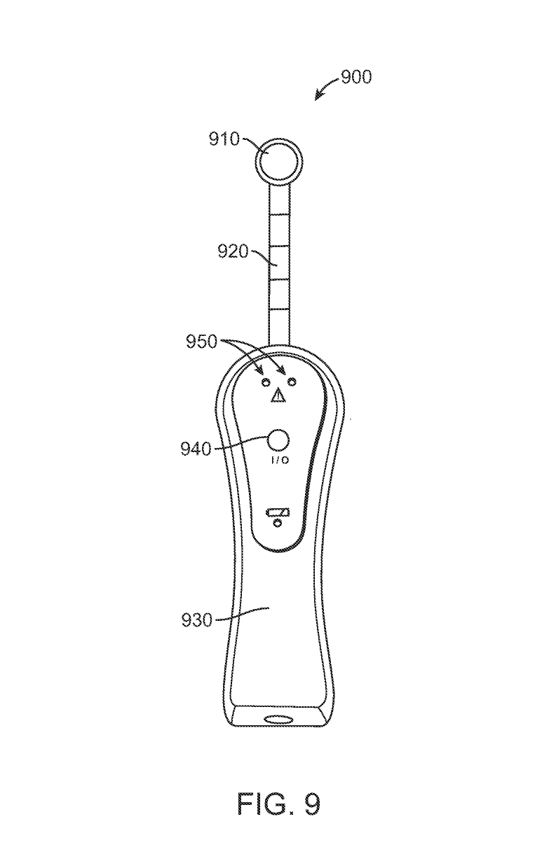

FIG. 9 is a diagram of a handheld device 900 for detecting the IPMC functionality of an in-vivo implant according to one embodiment of the present invention. As shown, a detector 910 is disposed at one end of an elongated member 920. The elongated member 920 is connected at its opposite end to a base 930. In operation, the handheld device 900 is inserted into a patient's mouth such that the detector 930 is positioned at/near the site of the implant. The device is activated by pressing a button 940 on the base 930. When testing is complete, a status indicator 950 with two light emitting diodes is activated and signals whether a normal response (green) or a potential problem (red) is detected in the IPMC material.

Handheld device 900 can be an embodiment of testing device 800 and can include power transmitter, data collection interface, processor, and user interface elements as previously discussed. For example, the detector 910 can include both an induction coil for transmitting power to the implant and a probe or sensor for measuring the electrical response characteristic. The processor can be disposed within base 930 and can analyze the sensor data and drive the user interface elements accordingly.

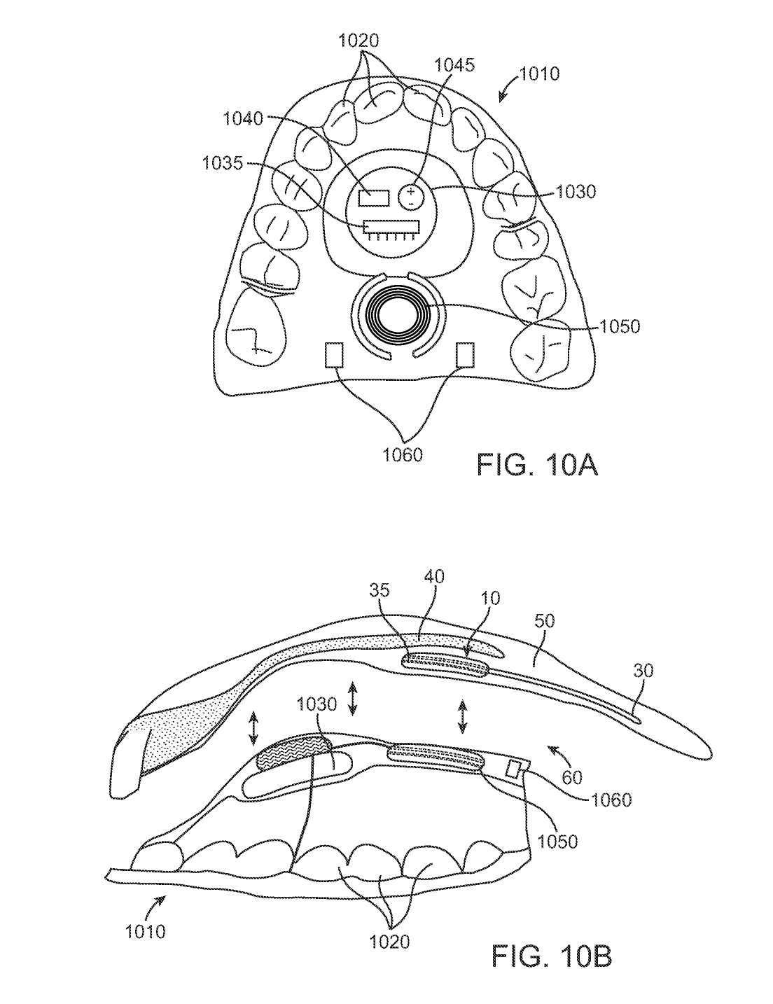

FIG. 10A is a diagram showing a retainer 1010 for detecting a functionality of an IPMC implant within the human body. As illustrated, retainer 1010 is adapted to be worn by a patient and includes indentations 1020 for engaging with the teeth and maintaining its position within the mouth 60. Controller 1030 can include a processor 1035 for detecting functionality of an IPMC airway implant device, a memory 1040 for storing data, and a power source 1045 such as a battery. The power source 1045 can be coupled to an induction coil 1050 and configured to source power to the implant device during testing operations. Also, a pair of sensors 1060 can be included for gathering data relating to the IPMC material's electrical response. When worn by the patient, sensors 1060 are positioned near the deformable portion of the airway implant which includes the IPMC material.

FIG. 10B shows the retainer 1010 of FIG. 10A in relation to the in-vivo IPMC implant 10. As illustrated, when retainer 1010 is worn by the patient, sensors 1060 are positioned near deformable portion 30 which includes the IPMC material. Processor 1035 can be configured to perform a testing method similar to that described in connection with FIG. 6. For example, the processor can control power delivery to the implant device 10 in first and second testing intervals which are separated by a continuous powering interval.

In one embodiment, sensors 1060 are configured to directly measure the DC voltage field surrounding the airway implant 10 and to communicate measurement data to the processor 1035. Alternatively, sensors 1060 can receive measurement data collected by the implant device 10. For example, sensors 1060 can include photodetectors configured to receive measurement data in a communication signal from the implant. In still other embodiments, sensors 1060 are omitted and the processor 1035 detects implant functionality by measuring current flow from the power supply 1045 during the testing intervals. In still another embodiment, the processor 1035 detects modulation of the RF energy at the induction coil 1050 such as can result from load changes at the implant. In each case, data indicative of the IPMC electrical response can be stored in memory 1040 for subsequent analysis.

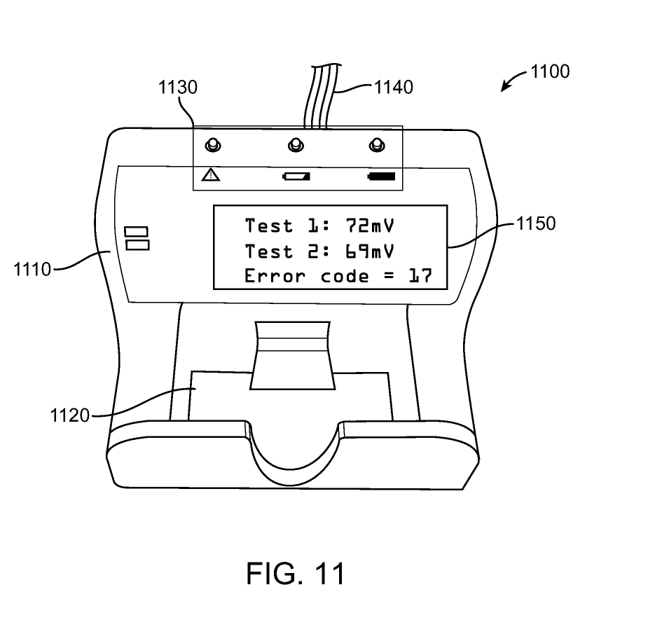

FIG. 11 shows a base unit 1100 for processing measurement data collected by the retainer of FIG. 10. As illustrated, base unit 1100 includes a housing 1110 with a recessed area 1120. Recessed area 1120 is adapted to receive retainer 1010. When the retainer 1010 is placed into the recessed area 1120, electronics within the base unit 1100 retrieve and analyze data stored in memory 1040. Optionally, base unit 1100 includes a ribbon cable 1140 or the like for interfacing with a computer.

In some embodiments, status indicators 1130 provide information about the functionality of the IPMC material based upon the retrieved data. For example, a green LED can signify that normal function of the IPMC material is detected, whereas a red LED can indicate problems. A yellow LED can signal improper alignment of the retainer 1010 or a general communication failure between the base unit and retainer. A display panel 1150 can provide additional information relating to the functionality of the implant such as measured values of the DC voltage field, diagnostic codes that can indicate a specific problem type, statistical values, etc.

As will be understood by those skilled in the art, the present invention may be embodied in other specific forms without departing from the essential characteristics thereof. Those skilled in the art will recognize, or be able to ascertain using no more than routine experimentation, many equivalents to the specific embodiments of the invention described herein. Such equivalents are intended to be encompassed by the following claims.

What is claimed is:

1. A method of testing an ionic polymer-metal composite device implanted within a patient's body, comprising:
supplying power to the device in a first testing interval;
measuring an electrical response of the device in the first testing interval;
supplying power to the device continuously following the first testing interval;
supplying power to the device in a second testing interval after the predetermined time;
measuring the electrical response of the device in the second testing interval; and
detecting a functionality of the device based on the electrical response measured in the first and second testing intervals.

2. The method of claim 1, wherein supplying power in the first and second testing intervals comprises generating a series of pulses.

3. The method of claim 1, wherein measuring the electrical response comprises detecting a voltage within the patient's body proximate to the device.

4. The method of claim 1, further comprising indicating the functionality of the device when the electrical response measured in the first testing interval is larger than the electrical response measured in the second testing interval.

5. The method of claim 1, wherein the electrical response is associated with a capacitance of the IPMC device.

6. The method of claim 1, wherein power is withheld from the device for a predetermined period before the first testing interval.

7. The method of claim 1, wherein the device is powered continuously for at least one minute following the first testing interval.

8. The method of claim 1, wherein measuring the electrical response comprises taking a series of measurements.

9. The method of claim 8, further comprising detecting an abnormality of the device based on the series of measurements.

10. A device for testing an ionic polymer-metal composite implant, comprising:
a transducer configured to supply power to the implant when the implant is within a patient's body;
a detector configured to measure an electrical response of the implant within the patient's body; and
a controller coupled to the transducer and the detector for controlling their respective operations, wherein the controller is configured to perform a test of the implant by supplying power to and measuring the electrical response of the implant in a testing interval, and the controller is configured to detect a functionality of the implant by performing a first test, powering the implant continuously, and performing a second test after the implant is continuously powered.

11. The device of claim 10, wherein the transducer supplies power to the implant through a series of pulses in the testing intervals.

12. The device of claim 10, wherein the detector is configured to measure a voltage level in a vicinity of the implant.

13. The device of claim 10, wherein the controller detects the functionality based on a comparison of the electrical response in the first test and the electrical response in the second test.

14. The device of claim 13, further comprising a user interface, and wherein the controller is configured to signal the functionality of the implant at the user interface when the electrical response of the implant in the first test is larger than the electrical response of the implant in the second test.

15. The device of claim 10, wherein the electrical response is associated with a capacitance of the implant.

16. The device of claim 10, wherein the controller is configured to power the implant continuously for at least one minute.

17. The device of claim 10, wherein the controller is configured to obtain a series of measurements in each testing interval.

18. The device of claim 17, wherein the controller is configured to detect an abnormality of the implant based on the series of measurements.

19. The device of claim 10, further comprising a mouthpiece adapted to be worn in the patient's mouth, wherein the transducer, detector and controller are attached to the mouthpiece.

20. The device of claim 10, further comprising an elongated body adapted to be inserted into a patient's mouth and wherein the detector is attached to a distal end of the elongated body.
